(19) United States
(12) Patent Application Publication
(10) Pub. No.: US 2006/0089512 A1
(43) Pub. Date: Apr. 27, 2006

(54) Method for reducing mehq content in acrylic acid

(75) Inventors: Andrea Karen Bennett, Cholburi (TH); Jurgen Ciprian, Ludwigshafen (DE); Mark Elliot, Ludwigshafen (DE); Kai Michael Exner, Eppelheim (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: BASF AKTIENGESELLSCHAFT, Ludwigshafen (DE)

(21) Appl. No.: 10/535,120

(22) PCT Filed: Dec. 3, 2003
(86) PCT No.: PCT/EP03/13621

§ 371 Date: May 16, 2005

(30) Foreign Application Priority Data
Dec. 6, 2002 (DE) 102 57 397

(51) Int. Cl. C07C 51/42; C07C 51/47

(52) U.S. Cl. 562/600

(57) ABSTRACT

The invention relates to the reduction of the content of MEHQ (methoxyhydroquinone) of acrylic acid or its salts by means of continuous adsorption on activated carbon. In particular, MEHQ is removed from partly or fully neutralized acrylic acid.

The invention relates to the reduction of the content of MEHQ (methoxyhydroquinone) of acrylic acid or its salts by means of continuous adsorption on activated carbon. In particular, MEHQ is removed from partly or fully neutralized acrylic acid.

MEHQ is used as a stabilizer of acrylic acid and its salts. In general, the stabilizer is added between 10 and 1000 ppm, and concentrations of between 50 ppm and 200 ppm are customary. Stabilization is necessary in particular when the acrylic acid has to be transported and/or stored between preparation and processing.

When the acrylic acid and/or its salts are processed to superabsorbents, the customer sectors desire a superabsorbent which does not have yellowish coloration. The main customer is the hygiene industry (diapers, adult incontinence articles, sanitary napkins, etc.) and the end user desires substantially white, “clean” products. When the polymerization of acrylic acid and its salts results in superabsorbent having yellow coloration, this does not meet the requirements of the end user.

It has been found that MEHQ is a cause of the yellow coloration of superabsorbents. This is especially true in combination with certain polymerization initiators which have oxidizing action.

JP 62106052 describes the removal of hydroquinones and other impurities from the reaction product of (meth)acrylic acid with alcohol using aqueous alkali solution.

JP 08310979 describes the removal of polymerization inhibitors from vinyl monomer systems by adsorption on, among other materials, alumina, silica gel, molecular sieves, activated carbon, ion exchange resins, chelating resins, zeolites and acidic clay.

It has been found that, surprisingly, the concentration of MEHQ can be particularly efficiently reduced in a continuous process using activated carbon. The acrylic acid should be from 75% to 105% neutralized. Neutralizing agents may be any customary bases, in particular alkali metal or alkaline earth metal hydroxides, such as NaOH or KOH; carbonates or hydrogen carbonates of alkali metal or alkaline earth metal salts; or else ammonia and ammonium salts. Preference is given to acrylic acid which has been from 90% to 103% neutralized, particular preference to acrylic acid which has been from 95% to 101% neutralized, in particular acrylic acid which has been from 98% to 100% neutralized.

The continuous adsorption in the process according to the invention is preferably carried out on a fixed bed.

Fixed bed refers to a dumped bed of activated carbon which remains substantially stationary while the acrylic acid flows through the fixed bed.

Continuous refers to a process in which, apart from the startup or shutdown of the process, as much acrylic acid is supplied as is removed.

Preference is given to a process according to the invention in which the continuous adsorption is carried out in one or more columns, in particular in one or two columns, which have been filled with activated carbon.

The following model for the adsorption behavior would be conceivable. During adsorption in one column, the first section of the column is saturated with the MEHQ, while the lower section is still free of MEHQ. Between these sections there is a mass transfer zone in which most of the MEHQ adsorption takes place. As soon as the first section of the column is saturated, the mass transfer zone moves downward through the activated carbon bed and may be regarded as an adsorption wave. Surprisingly, addition adsorption still takes place in the apparently saturated section even after a relatively long time (cf. table 3).

In general, the acrylic acid throughput of the solution to be depleted is predetermined, for example, by the plant capacity for superabsorbens. The maximum carbon loading with MEHQ may be obtained by optimizing the contact time between the acrylic acid with the MEHQ on the one hand and the activated carbon on the other hand. This is achieved by using broad columns in which the solution flow rate (volumetric flow rate per unit of column cross section) is slow. The mass transfer zone therefore moves slowly through the column. Moreover, two or more columns can be used in series, in order to extend the effective depth of the carbon bed. When a plurality of columns is used, this also allows exchange of a saturated column without leading to downtimes in the depletion of MEHQ.

The temperature of the process according to the invention is preferably between 0° C. and 30° C., in particular between 3° C. and 20° C.

In the process according to the invention, the activated carbon is preferably in a particle size distribution in which the average particle diameter is greater than 300 μm, preferably greater than 400 μm, in particular greater than 500 μm. These particle sizes are particularly suitable for a continuous process, since the activated carbon can easily be kept separate from the MEHQ-depleted acrylic acid. The activated carbon is typically in granulated form. Preferably 80% by weight, more preferably 90% by weight, in particular 95% by weight, of the activated carbon has a particle size between 350 μm and 1800 μm, in particular between 420 μm and 1700 μm.

In the process according to the invention, the activated carbon has preferably been acid-treated.

In the process according to the invention, preference is given to using activated carbon which has a high specific surface area (>600 m²/g, preferably >800 m²/g), in particular activated carbon having a specific surface area of from 900 to 1100 m²/g.

In the process according to the invention, activated carbon having a density between 400 g/l and 500 g/l is generally used.

Preferred types of activated carbon are CPG, granulated activated carbon from Calgon Carbon, Epibon MC-h 12X40 and Alcarbon WG 8X30, each from Elf Atochem, and ROW 0.8 Supra, granulated activated carbon from Norit.

In the process according to the invention, preference is given to reducing the concentration of MEHQ in acrylic acid by at least 50% or else at least 55%, at least 60%, at least 65%, at least 70%, preferably at least 75% or at least 80%, at least 85%, more preferably at least 90% or else at least 92%, at least 94%, in particular at least 95% or at least 96%, at least 97%, at least 98%, at least 99%, or even at least 99.5% or at least 99.6%, at least 99.7%, at least 99.8%, at least 99.9%. Preferred activated carbon can take up a maximum at equilibrium of at least 10 g, more preferably at least 11 g, particularly preferably at least 12 g, in particular at least 13 g, of MEHQ per 100 g of activated carbon.

The invention further relates to a process for preparing superabsorbents, including the step of optionally combining neutralized acrylic acid whose MEHQ content has been reduced according to the aforementioned process according to the invention with less neutralized, in particular nonneutralized, acrylic acid, subsequently polymerizing and optionally surface postcrosslinking. According to the invention, the superabsorbents prepared in this way can be used in hygiene articles.

In this context, superabsorbents are polymers based on acrylates which form water-insoluble hydrogel and have a centrifuge retention (CRC, measured according to the industrial standards of EDANA) of at least 15 g/g.

The reduction according to the invention of the MEHQ concentration in the solution to be polymerized enables the concentration of polymerization initiators to be reduced. This leads to less yellowing of the superabsorbent. In the case of MEHQ concentrations of 100 ppm or less, the polymerization initiator, for example sodium persulfate, can be dispensed with. In this case, the coloration is reduced even further. In addition, the proportion of extractables (16 h) in the superabsorbent is reduced.

### EXPERIMENTAL SECTION

Adsorption Isotherms

The adsorption isotherms can be used in a relatively simple manner by the Freundlich equation to determine the equilibrium concentration C of MEHQ and the value of the loading of MEHQ (X) per unit of activated carbon mass (M) by a double logarithmic plot.

The following equation applies:
X/M=kC^{1/n}
and therefore
Log X/M=log k 1 1/n log C
where k, n are constants.

The amount of MEHQ remaining in the solution was determined by HPLC.

Experiments on the Column—Continuous Method

“Breakthrough” curves were determined under dynamic conditions. Columns having an internal diameter of 20 mm and a length of 50 cm were filled with 81.7 g of adsorption material (activated carbon, inter alia). The average flow rate was 588 ml/h. The MEHQ content of the solution which had flowed through was determined. The “breakthrough” was defined at a value greater than 5 ppm.

Results

Adsorption Isotherms (Table 1)

The following adsorbents were tested at 15° C. using 100% neutralized acrylic acid.

TABLE 1

| | |
|---|---|
| Max. loading | Loading at 38 ppm of MEHQ |

| | |
|---|---|
| Adsorbent type | (g of MEHQ/100 g of adsorbent) |

| | | |
|---|---|---|
| CPG Carbon | 13.35 | 7 |
| ROW 0.8 Supra | 12.33 | 5 |
| F200 | 0 | — |
| Epibon | 10.95 | 7 |
| Alcarbon | 11.07 | 7 |

Experiments on the Column—Continuous Method (Tables 2 and 3)

Continuous experiments were carried out using CPG Carbon in the column. The residual content of MEHQ was determined at different times.

Table 2 shows the test results with CPG (Calgon) and with CECA BGX from Elf Atochem.

The maximum loadings are very similar (10.8 g of MEHQ and 10.6 g of MEHQ/100 g of activated carbon), but the density of CECA BGX is only half as high as that of CPG Carbon, which can therefore be used more cost-effectively. Under dynamic conditions (up to “breakthrough” of 5 ppm), the loading falls (10.8 g versus 7 g/100 g of activated carbon), but the pressure drop through the column remains constant. There is thus no polymerization to a significant extent. Temperature dependencies and the dependence on the degree of neutralization (adjusted using NaOH) were also determined. The 100% neutralized product obtained by the process according to the invention, if it contains little (<5 ppm) or virtually no MEHQ, can be stored under air for a limited period and under nitrogen over long periods of time.

When the acrylic acid solutions are used to prepare superabsorbents, distinctly reduced coloration is detected in comparison to acrylic acid solution which has not been depleted of MEHQ.

Temperature, Degree of Neutralization and Time Dependence of the Isotherms

Experiments were carried out at different temperatures, stir-in times, and with different degrees of neutralization (100%, 75%, 0%). The experiments were carried out using activated carbons CPG and CECA BGX.

TABLE 2

| | |
|---|---|
| | Loading at 38 ppm |
| | of MEHQ (g of |
| Degree of | MEHQ/100 g |
| neutralization in | of adsorbent) |

| solution | Temperature/time | CPG | CECA BGX |
|---|---|---|---|
| 100% | 15° C./24 h | 7 | — |
| 75% | 15° C./24 h | 1.7 | 1.5 |
| 75% | 25° C./48 h | 3.6 | 1.8 |
| 75% | 40° C./24 h | 2.3 | 1.8 |
| 0% | 15° C./24 h | 0.35 | 0.2 |
| 0% | 25° C./48 h | 2.3 | 0.4 |
| 0% | 40° C./24 h | 1 | 0.4 |

At 110% neutralization, only moderate adsorption was likewise determined.

In table 3, the dependence of the adsorption on the stirring time for one type of activated carbon (Epibon) is determined.

TABLE 3

| Degree of | | | |
|---|---|---|---|
| neutralization in | Stirring | Maximum loading | At 38 ppm of MEHQ |
| solution | time | (g of MEHQ/100 g of adsorbent) | |
| 100% | 24 h | 8.3 | 2.5 |
| 100% | 72 h | 13.7 | 3.4 |
| 100% | 144 h | 15 | 4.5 |

Preference is therefore given to processes according to the invention which lead to a long contact time between neutralized acrylic acid and activated carbon. The temperature can be kept low (from 0 to 10° C., for example 5° C.), in order to minimize any polymerization.

Superabsorbent Examples

Standard superabsorbent (see, for example, EP 372 706 p. 6 and 7, WO 99/42494, p. 4 to 8, or WO 01/38402) can also be prepared by the following formulation:

Comparative Example:

Monomer Solution

| | |
|---|---|
| Acrylic acid | 24.1% |
| (200 ppm of | |
| MEHQ) | |
| Water | 55.7% |
| 50% NaOH | 20.1% |
| PEGDA 400 | 0.62% by weight, based on acrylic acid |
| (Sartomer 344) | |
| Darocur | 0.036% by weight, based on acrylic acid |
| Sodium persulfate | 0.072% by weight, based on overall monomer solution |

The basis polymer obtained is comminuted, dried and surface postcrosslinked with 2-oxazolidone according to WO 99/42494.

Example:

As comparative example, except that the monomer solution consists of 75% neutralized acrylic acid solution which contains 50 ppm of MEHQ (prepared from 0 ppm MEHQ, 100% neutralized acrylic acid by the process according to the invention and 200 ppm MEHQ acrylic acid) and also 0% by weight of sodium persulfate is used.

Analytical Methods

The determination of the 16 h extractables was carried out according to ISO/DIS 17190-10. The color numbers were determined according to DIN 5033 (see also R. S. Hunter, The measurement of Apearamee, Wiley New York 1975). A Hunterlab LS 5100 colorimeter was used.

Result:

| Superabsorbent | | |
|---|---|---|
| according to | Extractable (16 h) | Color number b (DIN 5033) |
| Comparative example | 13% | 12 |
| Example | 5% | 8 |

1. A process for reducing a concentration of MEHQ in acrylic acid which has been from 75% to 105% neutralized, by a continuous adsorption on activated carbon.

2. The process of claim 1 wherein the acrylic acid has been from 90% to 103% neutralized.

3. The process of claim 1 wherein the acrylic acid has been from 95% to 101% neutralized.

4. The process of claim 1 wherein the acrylic acid has been from 98% to 100% neutralized.

5. The process of claim 1 wherein the continuous adsorption is carried out on a fixed bed.

6. The process of claim 1 wherein the continuous adsorption is carried out in one or more columns filled with activated carbon.

7. The process of claim 1 which is carried out at a temperature between 0° C. and 30° C.

8. The process of claim 1 wherein 90% by weight of the activated carbon has a particle size between 350 μm and 1800 μm.

9. The process of claim 1 wherein the activated carbon has been acid-treated.

10. The process of claim 1 wherein the activated carbon has a specific surface area of from 900 to 1100 m²/g.

11. The process of claim 1 wherein the activated carbon has a density between 400 g/l and 500 g/l.

12. The process of claim 1 wherein the concentration of MEHQ in the acrylic acid is reduced by at least 50%.

13. A process for preparing a superabsorbent comprising a step of optionally combining 75% to 105% neutralized acrylic acid whose MEHQ content has been reduced by continuous adsorption on activated carbon with a less neutralized acrylic acid subsequently polymerizing the acrylic acid, and optionally surface postcrosslinking the resulting superabsorbent.

14. (canceled)

15. The process of claim 1 which is carried out at a temperature between 3° C. and 20° C.

16. The process of claim 1 wherein the concentration of MEHQ in the acrylic acid is reduced by at least 75%.

17. The process of claim 1 wherein the concentration of MEHQ in the acrylic acid is reduced by at least 90%.

18. The process of claim 13 wherein the less neutralized acrylic acid is unneutralized acrylic acid.

19. A hygiene article comprising a superabsorbent prepared according to the process of claim 13.
